(19) United States
(12) Patent Application Publication
(10) Pub. No.: US 2011/0130969 A1
(43) Pub. Date: Jun. 2, 2011

(54) Resonant-Wavelength Measurement Method For Label-Independent Scanning Optical Reader

(76) Inventors: Jacques Gollier, Painted Post, NY (US); Gordon Michael Shedd, Lawrenceville, PA (US)

(21) Appl. No.: 12/952,970

(22) Filed: Nov. 23, 2010

Related U.S. Application Data
(60) Provisional application No. 61/264,938, filed on Nov. 30, 2009.

(51) Int. Cl. G01N 33/48

(52) U.S. Cl. 702/19

(57) ABSTRACT

A method of measuring a resonant wavelength of a resonant waveguide (RWG) biosensor in an array of RWG biosensors supported by a microplate in a label-independent optical reader is disclosed. An exemplary method includes scanning a light spot over the RWG biosensor to obtain a plurality of spectra from both a central portion and at least one edge portion of the RWG biosensor. The method includes calculating a weighted-average spectrum for the biosensor by averaging the plurality of spectra while applying greater weight to the central portion than to the at least one edge portion. The method includes determining the resonant wavelength from the weighted-average spectrum. The resulting resonant wavelength measurement has substantially reduced noise and provides improved performance for label-independent scanning optical reader systems that use scanned optical beams.

### CLAIMING BENEFIT OF PRIOR FILED U.S. APPLICATION

This application claims the benefit of U.S. Provisional Ser. No. 61/264,938, filed on Nov. 30, 2009. The content of this document and the entire disclosure of any publication or patent document mentioned herein are incorporated by reference.

### CROSS-REFERENCE TO RELATED APPLICATIONS

The present application relates to U.S. Provisional Patent Application No. 61/231,446 filed on Aug. 5, 2009, and entitled “Label-independent optical reader system and methods with optical scanning”

### FIELD

The present disclosure relates to label-independent optical reader systems, and in particular relates to a method for measuring biosensor resonant wavelengths in an optical reader system having one or more scanned optical beams to interrogate one or more biosensors.

### BACKGROUND

Certain optical reader systems use one or more scanned optical beams to interrogate a resonant waveguide grating biosensor to determine if a biomolecular binding event (e.g., binding of a drug to a protein) occurred on a surface of the biosensor. When an optical beam is scanned over a resonant waveguide (RWG) biosensor in two dimensions, certain variations can occur in the resonant wavelength measurement. Optical reader systems would benefit from improved resonant-wavelength measurement methods that account for such variations.

### SUMMARY

An aspect of the disclosure includes a method of measuring a resonant wavelength of a RWG biosensor in an array of RWG biosensors supported by a microplate in a label-independent optical reader. The method includes scanning a light spot over the RWG biosensor to obtain a plurality of spectra from a central portion and from at least one edge portion of the RWG biosensor. The method also includes an apodization filter process, including for example, calculating a weighted-average spectrum for the biosensor comprising averaging the plurality of spectra while applying greater weight to the central portion than to the at least one edge portion by greater than 5%. The method also includes determining the resonant wavelength from the weighted-average spectrum.

Another aspect of the disclosure includes method of calculating a resonant wavelength of a RWG biosensor having a central portion and at least one edge portion, based on a set of measured spectra obtained by scanning the RWG biosensor with a light beam and processing the reflected light. The method includes determining an average position of the RWG biosensor. The method also includes calculating a weighted-average spectrum for the biosensor by averaging the set of spectra while applying a weighting function centered on the average position, the weighting function weights the central portion greater than the at least one edge portion by greater than 5%. The method also includes calculating the resonant wavelength from the weighted-average spectrum.

Another aspect of the disclosure includes a method of reducing noise in a calculated resonant wavelength of a RWG biosensor in an array of RWG biosensors supported by a microplate and each biosensor of the array being separated from any other biosensor by gaps. The method includes scanning the plurality of RWG biosensors and the gaps therebetween with an optical beam and collecting reflected light from the biosensors and from the gaps. The method also includes establishing a set of spectra for each scanned RWG biosensor by calculating the spectral power of the reflected light and setting a power threshold that defines edge locations of the RWG biosensors. The method further includes calculating a weighted-average spectrum for each RWG biosensor comprising averaging the set of spectra for each RWG biosensor and weighting a central portion of the RWG biosensor more than edge portions of the RWG biosensor by greater than 5%. The method also includes calculating the resonant wavelength from the weighted-average spectrum.

These and other aspects of the disclosure will be further understood and appreciated by those skilled in the art by reference to the following written specification, claims, and appended drawings.

### BRIEF DESCRIPTION OF THE DRAWINGS

A more complete understanding of the present disclosure may be had by reference to the following detailed description when taken in conjunction with the accompanying drawings wherein:

FIG. 1 is a generalized schematic diagram of an example optical reader system for carrying out the method of the disclosure;

FIG. 2 shows an exemplary biosensor array operably supported in regions or “wells” of a microplate, which in turn is held by a microplate holder;

FIG. 3 is an example plot of resonant wavelength λ_{R} (nm) vs. position (mm) across the biosensor;

FIG. 4 is an example plot of the peak amplitude (photon counts) versus spectrometer pixel location, which corresponds to wavelength;

FIG. 5 is a detailed schematic diagram of a single-channel embodiment of a scanning optical reader system;

FIG. 6 is a close-up schematic diagram of an example scanning optical system that includes a scanning mirror device, a fold mirror, and an F-theta focusing lens;

FIG. 7 is a plot of the measured resonant wavelength λ_{R} (pm) versus X and Y position (mm) (solid and dashed lines, respectively) of the incident optical beam light spot as measured on several biosensors;

FIG. 8 is a close-up view of an example biosensor showing an exemplary scan path of the incident optical beam light spot over the biosensor;

FIG. 9 is an example plot similar to that of FIG. 7 and shows experimental measurements of the positioning sensitivity as the same biosensors are scanned in two dimensions rather than one dimension;

FIG. 10 is an example plot similar to that of FIG. 9 and shows the resonant wavelength variation as a function of the tip and tilt of the microplate for several biosensor measurements;

FIG. 11 is a schematic close-up view of a portion of the scanning optical system for a single-channel optical reader system and illustrates another exemplary system alignment method that employs a beam splitter and a detector;

FIG. 12 illustrates an exemplary embodiment of a method of establishing the position of the microplate or biosensor by dithering the light spot about a biosensor edge;

FIG. 13 is a schematic close-up view of a portion of a scanning optical system similar to that of FIG. 11 and illustrating an exemplary fiber array used to provide the optical reader system with multiple channels;

FIG. 14 is a schematic close-up view of a portion of the optical reader system associated with an n-channel embodiment, showing n fibers leading to n spectrometers;

FIG. 15 is a schematic diagram of a dual-head optical reader system;

FIG. 16 is a schematic diagram of an exemplary configuration of the two scanning optical systems (right and left) suitable for use in the dual scanning optical reader system of FIG. 15;

FIG. 17 is schematic diagram similar to FIG. 15 that illustrates an exemplary embodiment of dual scanning optical reader system that uses only one set of one or more spectrometer units;

FIG. 18 is similar to FIG. 17 and illustrates a simplified example embodiment wherein the dual scanning optical reader system includes a single coupling device, a single light source and a single spectrometer unit;

FIG. 19 is a close-up view of a section of the microplate of FIG. 2, and shows an example zig-zag scan path for the light spot wherein the scan path includes the RWG biosensors and the gaps therebetween;

FIG. 20 is a plot of the measured detector power P, vs. the spectrum number i and shows a peak and valley pattern associated with the scan path such as shown in FIG. 19 that includes the RWG biosensors and the gaps therebetween;

FIG. 21 is an example two-dimensional gray-scale map of the resonant wavelength λ_{R} measured over an example RWG biosensor and illustrates the variation in resonant wavelength with position within the biosensor; and

FIG. 22 is a histogram of the % occurrence versus measurement noise (pm) in the calculated resonant wavelength for measurements made on 96 RWG biosensors, with the resonant wavelength calculated using the prior art data processing method (black bars) and the improved processing method of the present disclosure (white bars).

### DETAILED DESCRIPTION

Reference is made to embodiments of the disclosure, exemplary embodiments of which are illustrated in the accompanying drawings.

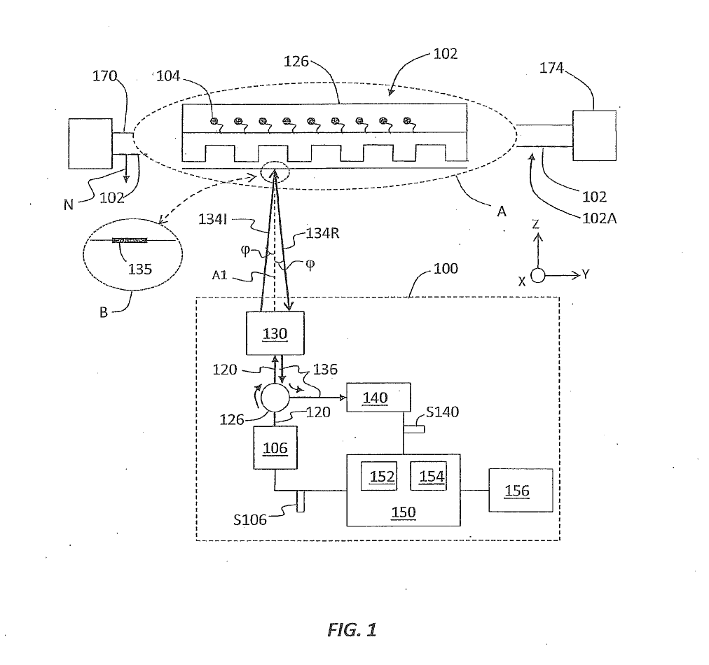

FIG. 1 is a generalized schematic diagram of an optical reader system (“system”) 100 suitable for carrying out the methods of the disclosure. System 100 is used to interrogate one or more biosensors 102 each having a surface 103 to determine if a biological substance 104 is present on the biosensor. Inset A shows a close-up of an exemplary biosensor 102. Biosensor 102 may be, for exemplary, a resonant waveguide grating (RWG) biosensor, a surface plasmon resonance (SPR) biosensor, or like biosensor.

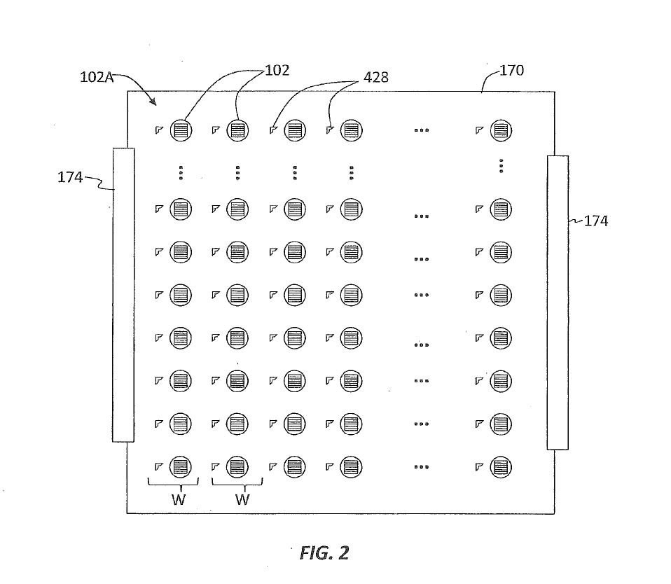

FIG. 2 shows an exemplary configuration where biosensors 102 are arranged in an array 102A and operably supported in regions or “wells” W of a microplate 170. An exemplary biosensor array 102A has a 4.5 mm pitch for biosensors 102 that are 2 mm square, and includes 16 biosensors per column and 24 biosensors in each row. Fiducials 428 that can be used to position, align, or both, the microplate 170 in system 100. A microplate holder 174 is also shown holding microplate 170. Many different types of plate holders can be used as microplate holder 174.

With reference again to FIG. 1, optical reader system 100 includes a light source assembly 106 (e.g., lamp, laser, diode, filters, attenuators, etc.) that generates light 120. Light 120 is directed by a coupling device 126 (e.g., a circulator, optical switch, fiber splitter or the like) to a scanning optical system 130 that has an associated optical axis A1 and that transforms light 120 into an incident optical beam 134I, which forms a light spot 135 at biosensor 102 (see inset B). Incident optical beam 134I (and thus light spot 135) is scanned over the biosensor 102 by the operation of scanning optical system 130. In prior art systems, the biosensor 102 is moved so the incident optical beam can be scanned across the biosensor 102. However, in the present disclosure, the incident optical beam 134I is scanned across a stationary biosensor 102 using scanning optical system 130, as described further below.

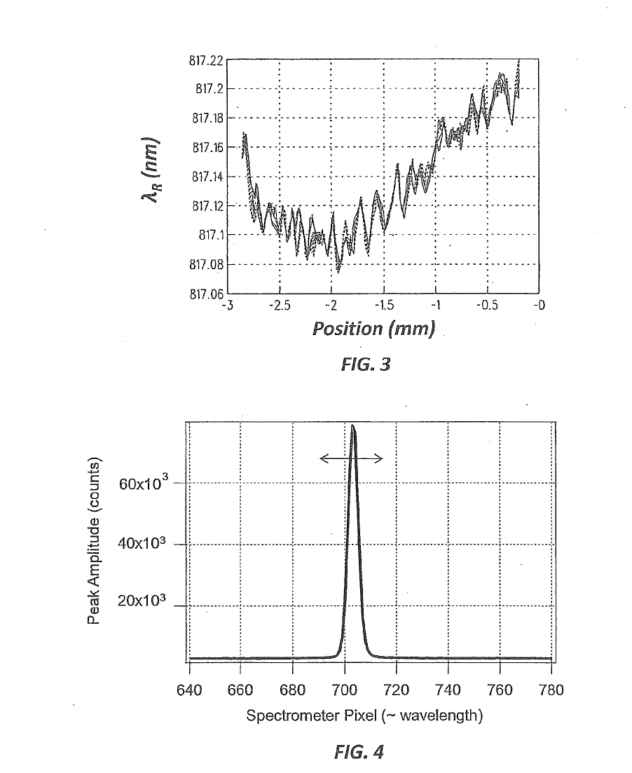

Incident optical beam 134I reflects from biosensor 102, thereby forming a reflected optical beam 134R. Reflected optical beam 134R is received by scanning optical system 130 and light 136 therefrom (hereinafter, “guided light signal”) is directed by coupling device 126 to a spectrometer unit 140, which generates an electrical signal S140 representative of the spectra of the reflected optical beam. In embodiments, a controller 150 having a processor unit (“processor”) 152 and a memory unit (“memory”) 154 then receives electrical signal S140 and stores in the memory the raw spectral data, which is a function of a position (and possibly time) on biosensor 102. Thereafter, processor 152 analyzes the raw spectral data based on instructions stored therein or in memory 152. The result is a spatial map of resonant wavelength (λ_{R}) data such as shown in FIG. 3, which shows the calculated resonance centroid as a function of the position of the scanning spot across the biosensor for a number of different scans. The variation of the resonance wavelength indicates if a chemical or biological reaction happened for a specific biosensor. In embodiments, controller 150 includes or is operably connected to a display unit 156 that displays measurement information such as spectra plots, resonant wavelength plots, and other measurement results, as well as system status and performance parameters. In embodiments, spectra can be processed immediately so that only the wavelength centroid is stored in memory 154.

Example biosensors 102 make use of changes in the refractive index at sensor surface 103 that affect the waveguide coupling properties of incident optical beam 134I and reflected optical beam 134R to enable label-free detection of biological substance 104 (e.g., cell, molecule, protein, drug, chemical compound, nucleic acid, peptide, carbohydrate) on the biosensor. Biological substance 104 may be located within a bulk fluid deposited on biosensor surface 103, and the presence of this biological substance alters the index of refraction at the biosensor surface.

To detect biological substance 104, biosensor 102 can be probed with incident optical beam 134I, and reflected optical beam 134R is received at spectrometer unit 140. Controller 150 can be configured (e.g., processor 152 is programmed) to determine if there are any changes (e.g., 1 part per million) in the biosensor refractive index caused by the presence of biological substance 104. In embodiments, biosensor surface 103 can be coated with, for example, biochemical compounds (not shown) that only allow surface attachment of specific complementary biological substances 104, thereby enabling biosensor 102 to be both highly sensitive and highly specific. In this way, system 100 and biosensor 102 can be used to detect a wide variety of biological substances 104. Likewise, biosensor 102 can be used to detect the movements or changes in cells immobilized to biosensor surface 103, for example, when the cells move relative to the biosensor or when they incorporate or eject material a refractive index change occurs.

If multiple biosensors 102 are operably supported as an array 102A in wells W of microplate 170, which in turn is supported by microplate holder 174, then they can be used to enable high-throughput drug or chemical screening studies. For a more detailed discussion about the detection of a biological substance 104 (or a biomolecular binding event) using scanning optical reader systems, reference is made to U.S. patent application Ser. No. 11/027,547. Other optical reader systems are described in U.S. Pat. No. 7,424,187, and U.S. Patent Application Publications Nos. 2006/0205058 and 2007/0202543.

The most commonly used technique for measuring biochemical or cell assay events occurring on RWG-based biosensors 102 is spectral interrogation. Spectral interrogation entails illuminating biosensor 102 with a multi-wavelength or broadband beam of light (incident optical beam 134I), collecting the reflected light (reflected optical beam 134R), and analyzing the reflected spectrum with spectrometer unit 140. An exemplary reflection spectrum from an example spectrometer unit 140 is shown in FIG. 4, where the “peak amplitude” is the number of photon counts as determined by an analog-to-digital (A/D) converter in the spectrometer. When chemical binding and like associations or interactions occur at biosensor surface 103, the resonance shifts slightly in wavelength as indicated by the double arrow, and such shift can be detected by spectrometer unit 140.

While the general concept of spectral interrogation of biosensor 102 is straightforward, the implementation details of how light can be delivered to and collected from the biosensor can have a major impact on the quality of the data and practical utility of system 100. For example, due to inevitable non-homogeneity of the resonant wavelength λ_{R} across biosensors 102, the measured resonant wavelength λ_{R} can be extremely sensitive to the position of incident optical beam 134I over the biosensor.

### Single-Channel Scanning Optical Reader System

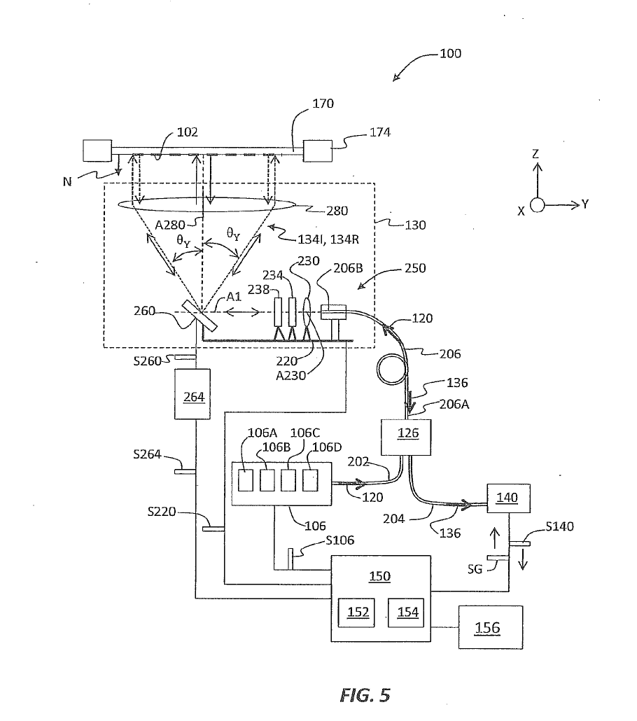

FIG. 5 is a detailed schematic diagram of a single-channel embodiment of system 100. Cartesian X-Y-Z coordinates are shown for reference. An exemplary light source assembly 106 comprises a light source 106A, a variable optical attenuator 106B, a polarization scrambler 106C and an optical isolator 106D. Polarization scrambler 106C serves to randomize the polarization of light 120, and optical isolator 106D serves to prevent scattered or reflected light from returning to light source 106A.

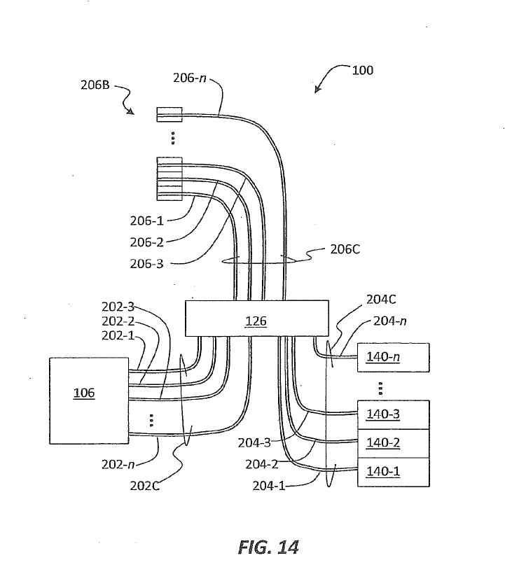

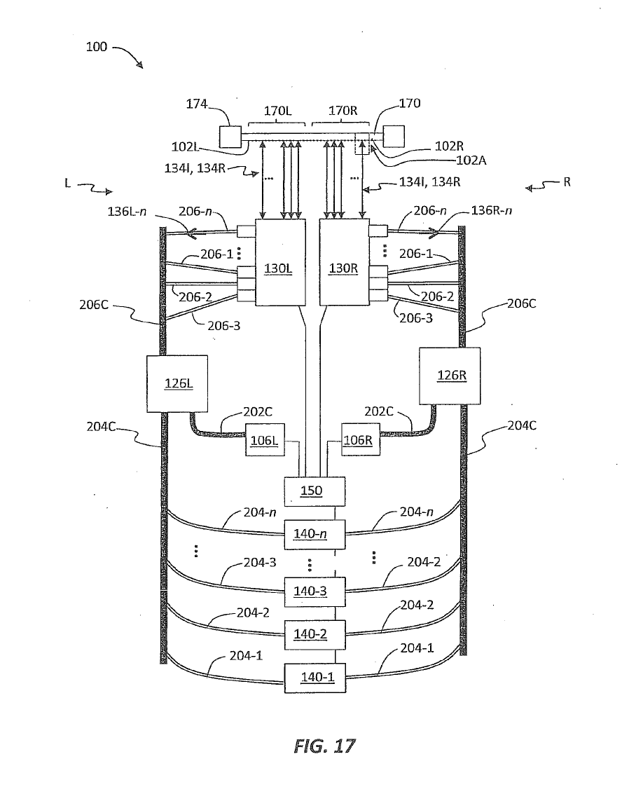

An exemplary light source 106A includes a wide spectrum source such as a superluminous diode (SLD). Light source assembly 106 is optically connected by a first optical fiber section 202 to coupling device 126, which in the present embodiment is a 1×2 fiber splitter. Spectrometer unit 140 comprises a spectrometer, such as an HR-2000 spectrometer, available from Ocean Optics, Dunedin, Fla. Spectrometer unit 140 can be connected by a second optical fiber section 204 to coupling device 126. A third optical fiber section 206 can be connected at one end 206A to coupling device 126, while the other end portion 206B can be mounted on a X-Y-Z translation stage 220. Also mounted on translation stage 220 can be a focusing lens 230 having a focal length f2, a linear polarizer 234 and a quarter-wave plate 238. Note that focusing lens 230 may comprise one or more optical elements. Fiber section end 206A, focusing lens 230, linear polarizer 234 and quarter-wave plate 238 constitute an adjustable beam-forming optical system 250 that shares the aforementioned optical axis A1. In embodiments, translation can be manually adjustable, while in other embodiments stage 220 can be adjustable under the control of controller 150 via a control signal S220. In embodiments, the first, second, and third fiber sections 202, 204 and 206 can be single-mode (SM) fiber sections. In exemplary embodiments discussed below, fiber sections 202, 204, and 206 can be carried by respective optical fiber cables 202C, 204C and 208C (see FIG. 14 and FIG. 17) that carry one or more of the respective fiber sections.

System 100 can also include a scanning mirror device 260 arranged along optical axis A1 adjacent beam-forming optical system 250. Scanning mirror device 260 can be, for example, a micro-electro-mechanical system- (MEMS)-based mirror, such as is available from Mirrorcle Technologies, Inc., Albany, Calif., or from Texas Instruments, Dallas, Tex., as model TALP 1011, for example. Other exemplary embodiments of scanning mirror device 260 can include a scanning galvanometer, a flexure-based scanning mirror, an oscillating plane mirror, a rotating multifaceted mirror, and a piezo-electric-driven mirror. Scanning mirror device 260 can be adapted to scan in at least one dimension (1D) and preferably two-dimensions (2D) (i.e., along axes X and Y, thereby defining associated scanning angles θ_{X} and θ_{Y}). Scanning mirror device 260 can be operably connected to a mirror device driver 264, which may be based on voltage or current depending on the nature of scanning mirror device 260. In embodiments, scanning mirror device 260 can be mounted on translation stage 220.

A field lens 280 can be arranged along optical axis A1 adjacent scanning mirror device 260 and opposite beam-forming optical system 250. In embodiments, field lens 280 has an F-theta configuration wherein light from any angle θ is directed substantially parallel to optical axis A1 (i.e., φ about 0°). Suitable F-theta field lenses 280 are commercially available from optics suppliers, such as Edmund Optics, Barrington, N.J. Field lens 280 has a focal length f1 and comprises at least one optical element. In embodiments, field lens 280 comprises multiple optical elements, including at least one mirror, or at least one lens, or a combination of at least one mirror and at least one lens. In embodiments, field lens 280 includes one or more aspherical surfaces.

System 100 also includes the aforementioned microplate holder 174 configured to operably support microplate 170, which in turn is configured to operably support an array of biosensors 102. In embodiments, the position of microplate holder 174 is adjustable so that the position of microplate 170 can be adjusted relative to optical axis A1. Scanning mirror device 260 is located at the focus of field lens 280, i.e., at a distance f1 from the field lens.

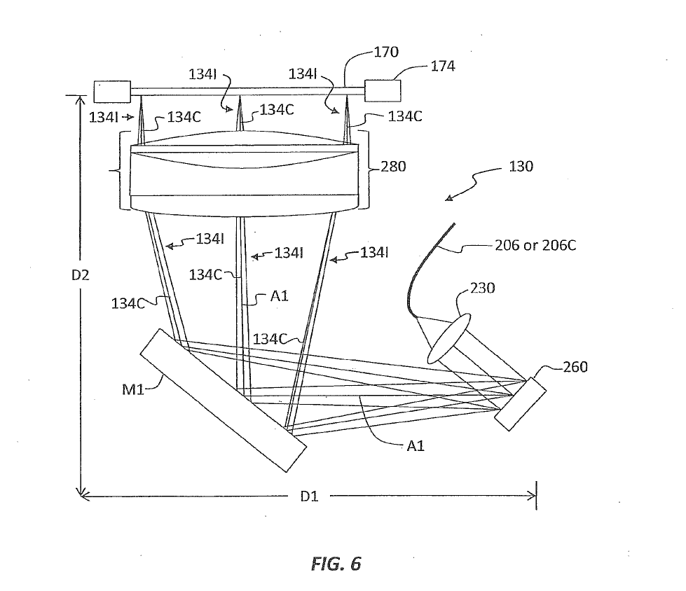

FIG. 6 is a close-up schematic diagram of an exemplary scanning optical system 130 shown optically coupled to beam-forming optical system 250 and that includes scanning mirror device 260, a fold mirror M1, and F-theta field lens 280. Also shown is microplate holder 174 with microplate 170 supported thereby. Fold mirror M1 can be used to fold optical axis A1 and thus fold the optical path to make scanning optical system 130 more compact. In embodiments, focusing lens 230 has a focal length f2=10 mm and field lens 280 has a focal length f1=200 mm with an aperture of 72 mm. This particular configuration for scanning optical system 130 fits within dimensions L1×2=140 mm×140 mm and thus has a relatively compact form factor. In embodiments, beam-forming optical system 250 can be included in scanning optical system 130.

The size of the microplate 170 that can be scanned by scanning mirror device 260 is given by the tangent of the mirror deflection multiplied by the focal length of the field lens 280. So, with +/−10 degrees of optical deflection and a 200 mm focal length field lens 280, a 72 mm area can be scanned in both the X- and Y-directions.

The exemplary scanning optical system 130 of FIG. 6 is capable of interrogating a single microplate column of biosensors 102 when configured in a standard microplate format of sixteen wells per column on a 4.5 mm pitch, or about a 72 mm total distance. An exemplary nominal size of light spot 135 formed by incident optical beam 134I at microplate 170 is 0.1 mm at 1/e² (diameter) and an exemplary beam diameter of the incident optical beam at scanning mirror device 260 is 2 mm at 1/e². FIG. 6 illustrates incident optical beam 134I at three different scan positions (angles). The central ray of incident optical beam 134I is denoted 134C. Note the incident optical beam 134I is a converging beam at microplate 170, with the central rays 134C being parallel to optical axis A1 at the microplate.

An exemplary scanning mirror device 260 is a MEMS-based mirror (such as the aforementioned TALP1011 from Texas Instruments) having a clear aperture of 3.2 mm×3.6 mm and optical scanning angles θ_{X} and θ_{Y} of +/−10°. The variation of incidence angle φ of incident optical beam 134R over microplate 170 due to aberrations in an exemplary field lens 280 was found in one example system 100 to be less than 0.3 mRd.

Controller 150 is operably connected to light source assembly 106, spectrometer unit 140 and mirror device driver 264, and is configured (e.g., via software embodied in a computer readable medium such as in processor 152 or memory 154) to control the operation of system 100 as described below. In embodiments, controller 150 can be configured with a General Purpose Interface Bus (GPIB) and the devices to which the controller is operably connected can be configured to communicate with the controller using the GPIB.

With reference again to FIG. 5, in the general operation of system 100, controller 150 sends a light source control signal S106 to light source assembly 106 to cause the light source assembly to generate light 120, which is coupled into first fiber section 202 as guided light. Light 120 travels down first fiber section 202 and to third fiber section 206 via coupling device 126. Light 120 is then processed by beam-forming optical system 250, which forms incident optical beam 134I. Incident optical beam 134I is then selectively deflected by scanning mirror device 260 under the operation of a control signal S260 from mirror device driver 264, which in turn is activated by a control signals S264 from controller 150. Because scanning mirror device 260 is located at the focus of field lens 280, in the region between the field lens and microplate, the incident optical beam 134I (or, more precisely, the central ray 134C of this beam) is parallel to optical axis A1 for all deflection angles 170. System 100 can be adjusted so that incident optical beam 134I remains substantially normal to microplate 170 as the beam scans the microplate.

Incident optical beam 134I scans over biosensor 104 as described below and reflects therefrom at substantially normal incidence to form reflected optical beam 134R. Reflected optical beam 134R thus travels substantially the reverse optical path of incident optical beam 134I and is coupled back via beam-forming optical system 250 into third fiber section 206 at end portion 206B and becomes guided light signal 136. Guided light signal 136 then travels through third optical fiber section 206 to second optical fiber section 204 via coupling device 126, where it is received and spectrally decomposed by spectrometer unit 140. Spectrometer unit 140 provides electrical signal S140 representative of the spectral information in reflected optical beam 134R to controller 150 and to memory 154 therein. Memory 154 stores the spectral information as a function of the scanning angles (θ_{X}, θ_{Y}). In embodiments, memory 154 stores and processor 152 runs analysis software for analyzing and visualizing the spectral information, such as Matlab, available from Mathworks, Inc., Natick, Mass.

In embodiments, memory 154 stores a number (e.g., 50) of spectra for each biosensor 102, and processor 152 sums the spectra to obtain a total spectrum, and then calculates the centroid to determine resonant wavelength λ_{R}. In embodiments, tens, hundreds, or thousands of spectra can be saved in memory 154 for processing by processor 152. Spectra measurements can be divided up by, for example, individual biosensors 102 or by columns or rows of biosensors.

### Biosensor Scanning

One method of scanning using system 100 is to operate scanning mirror device 260 to scan one or more biosensors 102 in a single scanning direction. However, a shortcoming of this approach is that the resonance wavelength λ_{R} varies significantly as a function of the position of light spot 135 across biosensor 102. Accordingly, in this approach the position of light spot 135 needs to be monitored closely to avoid introducing measurement bias.

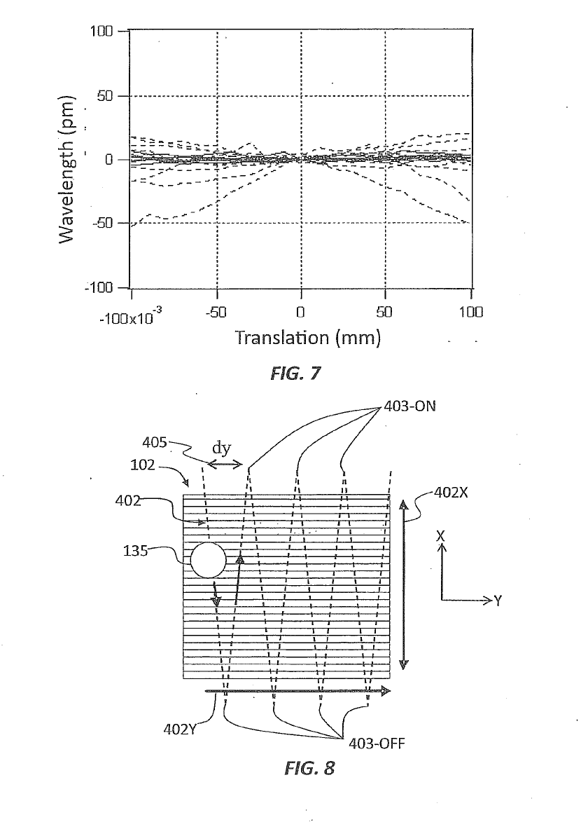

FIG. 7 is a plot of the measured resonant wavelength λ_{R} (pm) versus x and y displacement (mm) of biosensor 102 as measured on nine different biosensors. The solid line represents translation in the x-direction and the dashed line represents translation in the y-direction. During the measurement, optical beam 134I was scanned back and forth (dithered) across the entire biosensor length along the x-direction, but no movement of the optical beam was made in the y-direction. The spectra collected were integrated over a period longer than the back and forth scan time in the x-direction. When microplate 170 is moved perpendicular to the scan axis, a large amount of wavelength change can be observed (dashed lines). When microplate 170 is moved along the scan axis, almost no wavelength change is observed (solid lines). The lesser amount of wavelength change for the x-displacement is observed because, regardless of the biosensor displacement in x, the entire line scan across the biosensor grating is collected due to the x-dither applied to the beam. The plot also shows variations as large as 0.5 pm/micron, which means that measurement bias below 0.1 pm requires re-positioning errors to be lower than 0.2 micrometers, which is relatively difficult to achieve.

Accordingly, a particularly useful method of operating system 100 involves scanning biosensors 102 with incident optical beam 134I in two dimensions (x and y) to obtain an integrated measurement of each scanned biosensor. Because a MEMS-based mirror scanning device can be driven at a relative high frequency (e.g., >100 Hz), it is possible to rapidly perform such a two dimensional scan of a sensor. In one example, biosensor 102 is scanned by moving optical beam 134I (and thus light spot 135) faster in one of the two dimensions.

FIG. 8 is a close-up view of an example biosensor 102 and shows an exemplary scan path 402 of light spot 135 (or equivalently, incident optical beam 134I) over at least a portion of the biosensor. Scan path 402 has a scanning pitch dy and uses the Y-axis as the slow-scanning axis (i.e., y-direction scan path component 402Y) and the X-axis as the fast-scanning axis (i.e., x-direction scan path component 402X), which forms a zig-zag scan path. Rapid scanning of light spot 135 in such a manner allows a much larger “effective light spot” to be created, which can be made larger than biosensor 102. However, unlike creating a large incident optical beam 134I using optical magnification alone, the angular acceptance of the system is not reduced (angular acceptance being proportional to the inverse of the light spot diameter), and system flexibility is maintained to process reflected optical beam 134R from biosensor 102 with high spatial resolution.

An exemplary X-axis scanning rate is about 400 Hz. In an example embodiment, each X-axis scan pass in the +x direction corresponds to a scan reading wherein spectrometer 140 can be activated and processes guided light signal 136. Thus, at turn-around location T-ON in scan path 402, spectrometer 140 is triggered ON by gating or trigger signals SG from controller 150 and starts accumulating photons associated with guided light signal 136. At turn-around locations T-OFF in scan path 402, photon integration triggered off by trigger signal SG from controller 150 and spectrometer 140 sends via signal S140 a single spectrum into memory 54 for further processing by processor 52. As an example, spectrometer 140 is triggered at 400 Hz and the spectral integration time is about 1 millisecond. Assuming, for instance, that the scanning speed in the y-direction is such that light spot 135 scans the entire biosensor 132 within 0.5 seconds, system 100 collects 200 spectra (0.5 s*400 Hz) per biosensor, with each spectrum being integrated over the entire length of the biosensor along the Y-axis. Alternatively, the signal reflected by biosensor 102 can be integrated during the entire scan time that it takes for optical beam 134I to cover the biosensor in two dimensions. In this example, a single accumulated spectrum contains all of the information about a single measurement of the given biosensor.

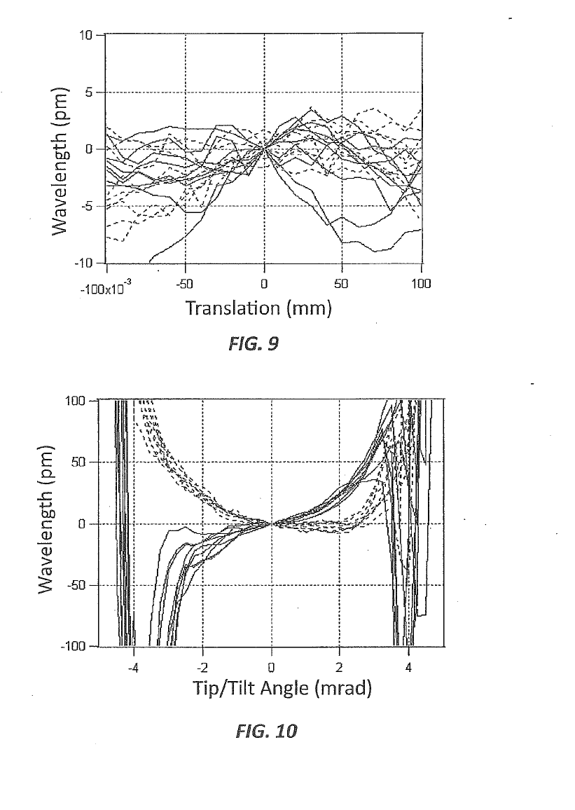

When using a scanning pitch dy smaller than the diameter of light spot 135, the effectively large beam allows the sensitivity to lateral misalignment to be dramatically reduced. FIG. 9 is a plot similar to FIG. 7 and shows experimental measurements of the positioning sensitivity as the same nine biosensors 102 are scanned in 2D rather than 1D. As can be seen, the lateral sensitivity can be reduced by at least a factor of five in the 2D scan over the 1D scan. This removes the need for precise repositioning of microplate 170. This in turn allows for less expensive microplate translation stages 174 to be selected to move microplates 170 in and out of system 100.

In embodiments, scan path 402 traversed by optical beam 134I comprises a zig-zag pattern, such as the sharp triangular wave depicted in FIG. 8, or a sinusoidal path by appropriately modulating the x-axis scan to prevent high frequencies from exciting the resonances of scanning mirror device 260. In embodiments, signal S260 from mirror device driver 264 is a step function combined (e.g., convolved) with a smoothing function (e.g., a Gaussian filter) to create a smoothed step function that avoids “ringing” or other adverse scanning effects that cause deviations in scan path 402 from a desired scan path as a result of driving scanning mirror device 260. In embodiments, incident optical beam 134I can be scanned in 2D (e.g., in a zig-zag fashion as described below) as the incident optical beam travels between biosensors 102 to avoid having to start and stop the scanning process, which can introduce undesirable scan path deviations. In an exemplar of this scanning method, controller 150 sends gating (or triggering) signals SG to spectrometer unit 140, wherein the gating signals timed so that the spectrometer unit only processes reflected optical beams 134R from biosensors 102 and not from the surface of microplate 170.

### Compensation of Microplate Misalignment

The aforementioned U.S. Pat. No. 7,424,178, shows that when using SM fiber sections 202-206, the resonance wavelength λ_{R} is not substantially affected by an angular misalignment. However, second order effects, such as lens aberrations or deviations of the profiles of incident and reflected beams 134I and 134R from a perfect Gaussian shape, can introduce some residual angular dependence on the measurement of the resonant wavelength λ_{R}.

FIG. 10 is a plot similar to FIG. 9 and shows the resonant wavelength variation as a function of the angular tip (solid line) and tilt (dashed line) of microplate 170 for several biosensor measurements. The curves are reasonably flat for tilt angles below +/−2 mrad, but significantly increase in slope beyond this limit. Precise angular repositioning can be accomplished using a three-point contact microplate holder 174 to provide positional repeatability to about 25 microrad. However, in commercial embodiments it may be desirable to use less expensive and less complicated microplate holders 174 that also have less angular precision. In instances where the angular repositioning of microplate 170 is worse than about 2 mrad, a method of positional compensation may be needed that provides easy realignment of the system.

It is noted that the curves in the plots of FIG. 9 and FIG. 10 are very repeatable from biosensor to biosensor. Thus, the shape of the curves is dictated by the optical aberrations present in the illumination system rather than by the biosensors themselves. Thus, in embodiments, the signal from a reference biosensor 102 is measured and then subtracted from the signal from the biosensors of interest, to remove wavelength shifts due to angular changes of microplate 170.

In embodiments, system 100 can be configured so that the position of field lens 280 is adjustable relative to scanning mirror device 260 and beam-forming optical system 250. In embodiments, the relative positions of field lens axis A280, scanning mirror device 260 and focusing lens axis A230 are adjustable, i.e., one or more of these elements is displaceable relative to optical axis A1. In embodiments, this adjustability is provided by translation stage 220. The angle of incidence φ of incident optical beam 134I relative to microplate 170 is defined by the vector joining the center of the incident optical beam at focusing lens 230 and the apex of field lens 280. In embodiments, incidence angle φ of incident optical beam 134I can be adjusted by adjusting the relative position of lenses 230 and 280. Such adjustment can be made, in embodiments, by adjusting translation stage 220 that includes scanning mirror device 260 and focusing lens 230. This operation does not require translation stage 220 to have high precision. By way of example, for a field lens 280 having a focal length f1=200 mm, the alignment precision only needs to be in the order of 0.2 mm to insure that the precision of incidence angle γ is within 1 mrad. This adjustability makes system 100 substantially insensitive to microplate misalignment.

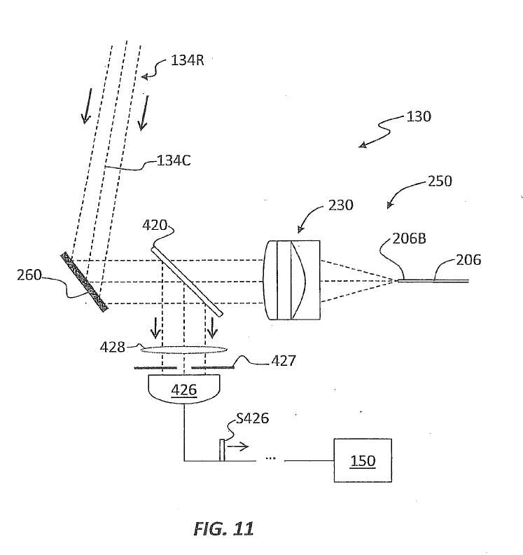

In embodiments, system 100 is aligned by optimizing the optical power coupled back into scanning optical system 130 prior to starting the scanning measurements. FIG. 11 is a schematic close-up view of a portion of scanning optical system 130 for a single-channel optical reader system 100 and illustrates another exemplary system alignment method. The alignment method employs a beam splitter 420 arranged in the optical path between beam-forming optical system 250 and scanning mirror device 260. Beam splitter 420 is configured to direct a portion of reflected optical beam 134R to a photodetector 426 that is laterally aligned with respect to the center of focusing lens 230. Photodetector 426 generates a photodetector signal S426 representative of the amount of optical power detected, and in embodiments, this signal can be directed to controller 150. Photodetector 426 can be, for example, a small-area photodiode, or a photodiode with a limiting aperture 427 in front, as shown in FIG. 11. A lens 428 (shown in phantom) may also be used to focus light onto photodetector 426 in the absence of limiting aperture 427, or in combination therewith. The alignment optimization can be performed by adjusting the position of focusing lens 230 relative to field lens 280 such that the light collected by the photodetector 426 is maximized. In embodiments, this maximization and adjustment process can be accomplished automatically under the operation of controller 150.

Alternatively, the photodetector 26 and limiting aperture 427 may be replaced by a position-sensitive diode or CCD camera. In this instance, the position of the reflected beam 134R on photodetector 26 is monitored, and the adjustment process entails moving the focusing lens 230 relative to the field lens 280 until the reflected beam spot is set to a pre-determined location on the photodetector.

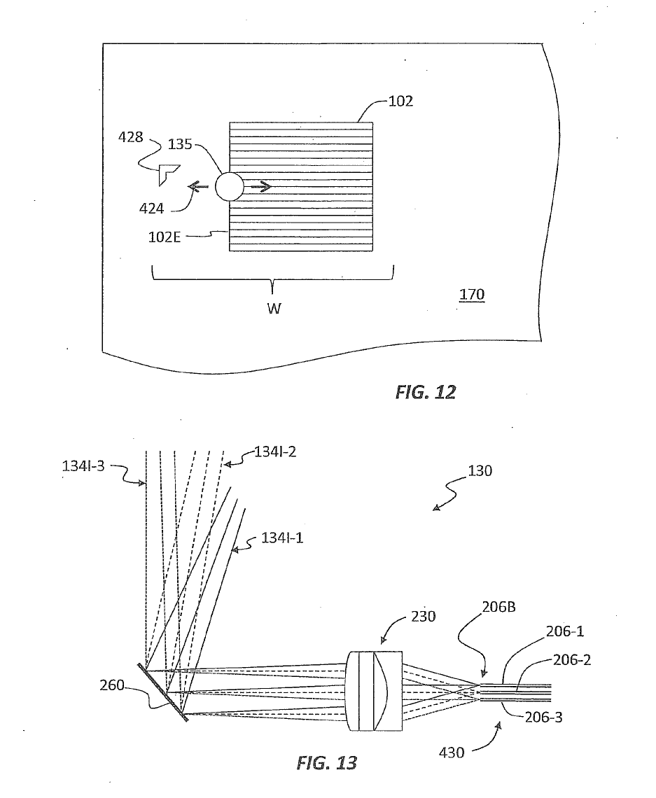

FIG. 12 illustrates an exemplary embodiment of a method of establishing a relative position of microplate 170 within system 100. The microplate position (or biosensor position) is established by directing light spot 135 to an edge 102E of biosensor 102 and then dithering the light spot position relative to the biosensor edge as illustrated by arrows 424. Photodetector 426 records the power of reflected optical beam 134R and symmetric power fluctuation is used to establish the biosensor edge location and thus the microplate position as well as the biosensor position on the microplate. Various edge detection algorithms can be applied to the photodetector signal in processor 152 to establish the position of biosensor edge 102E. The dithering of light spot 135 can be accomplished by a scanning mirror device 260 being driven in an oscillating manner by a mirror device driver 264.

In embodiments, fiducials 428 formed on microplate 170 are used to facilitate microplate alignment. In embodiments, light spot 135 is scanned over one or more fiducials 428 to establish the position of microplate 170. Other embodiments of systems and methods for aligning microplate 170 in system 100 using fiducials 428 are described, for example, in the aforementioned U.S. Patent Application Publication No. 2007/0202543.

### Multiple-Channel Scanning Optical Reader System

Experiments indicate that a significant limiting factor for the resolution of system 100 is optical shot noise. Shot noise can be reduced by collecting more photons for each biosensor measurement. Most often, the factor that limits the amount of photons that can be collected is the spectrometer unit 140. The number of photons that can be collected by each pixel in a linear detector array of a spectrometer is given by I=WD/T, where I is the maximum flux of photons that can be collected per second, T is the fastest integration or readout time of the spectrometer detector, and WD is the well depth of the detector, which sets the maximum number of photons that can be collected over the integration time without reaching the saturation threshold.

To increase the maximum detected photon flux to decrease the measurement noise, one can select a spectrometer detector that has deeper wells, or one can increase the speed at which the detector is read out. Another option uses multiple channels each having an associated fiber 206 and spectrometer unit 140. In this instance, the total collected photon flux is multiplied by the number of spectrometers (or “channels”) used.

An exemplary embodiment of system 100 provides for multiple measurement channels while employing a single scanning mirror device 260 by providing multiple fibers 206 arranged in an array 430 at the focus of focusing lens 230. FIG. 13 is a schematic close-up view of a portion of scanning optical system 130 illustrating an exemplary fiber array 430 having three fibers 206-1, 206-2 and 206-3 by way of illustration. The three fibers 206-1, 206-2 and 206-3 are disposed close to the focus of focusing lens 230 and emit respective optical beams 134I-1, 134I-2 and 134I-3 at different pointing angles. Any reasonable number of fibers 206 can be used to form array 430, with from two to about twelve fibers being particularly useful.

To first approximation, the pointing angle offset of incident optical beams 134I is given by θ=Dyf/f2, where θ is the pointing angle of the incident optical beams, Dyf is the position of an individual fiber with respect to optical axis A1, and f2 is the focal length of focusing lens 230. Hence, an array of optical beams 134I is directed to microplate 170, with the position separation of the beams at the microplate given by:

Dyp=θ*f1=Dyf*f1/f2

where Dyp is the separation between the incident optical beams at microplate 170 and f1 is the focal length of field lens 280.

By properly setting the pitch P of fiber array 430, the separation of light spots 135 associated with input optical beams 134I can be made to correspond to an integer number times the pitch P′ of biosensors 102 in biosensor array 102A. The separation of optical beams 134I at fiber ends 206B can be magnified by a factor of (f1/f2) at microplate 170 by the operation of lenses 230 and 280. As a consequence, the image of each fiber end 206B is centered on a specific biosensor 102 so that each fiber interrogates (illuminates) a different area of microplate 170. As an example, for f1=200 mm and f2=10 mm, and a biosensor array pitch P′ of 4.5 mm, the pitch P of fiber array 430 is 0.225 mm.

As scanning mirror device 260 scans, the array of optical beams 134I moves and the corresponding reflected optical beams 134R from each illuminated biosensor 102 are simultaneously collected by their respective fibers 206 as described above. In general, n fibers 206 can be used to form n-channels, where n is an integer is equal to or greater than 1. The guided light signal 136 in each fiber 206 is then routed to a corresponding spectrometer unit 140 (e.g., 140-1, 140-2, etc.), as illustrated in FIG. 14 for n different fibers 206 and n spectrometer units 140. Note that coupling device 126 becomes a n×2n coupling device in this configuration, with light source 106 being coupled to n fiber sections 202-1, 202-2, . . . 202-n, which sections may be configured in a optical fiber cable 202C. Fiber sections 204 and 206 can be also be configured in respective optical fiber cables 204C and 206C. In embodiments, the various multiple optical fiber sections can be combined into respective optical fiber ribbon sections or cables.

### Dual-Head Optical Reader System

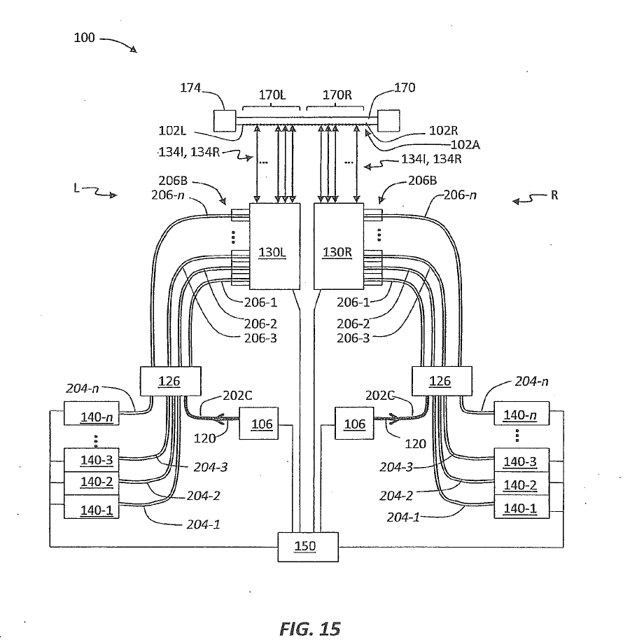

FIG. 15 is a schematic diagram of another exemplary embodiment of a “dual-scanning” multiple-channel optical reader system 100 that combines two of the single or multichannel systems described above. System 100 of FIG. 15 has “left” and “right” sides denoted L and R, and utilizes two scanning optical systems 130 (shown as 130L and 130R) that each interrogate sub-regions 170L and 170R of microplate 170 in a scanned fashion as described above in order to measure respective arrays 102A of biosensors 102 (see FIG. 1). Each of the two scanning optical systems 130L and 130R is shown configured in the multiple channel embodiment of system 100 described above, where one side of the system is essentially a reflection of the other, but is configured to operate under the control of a single controller 150.

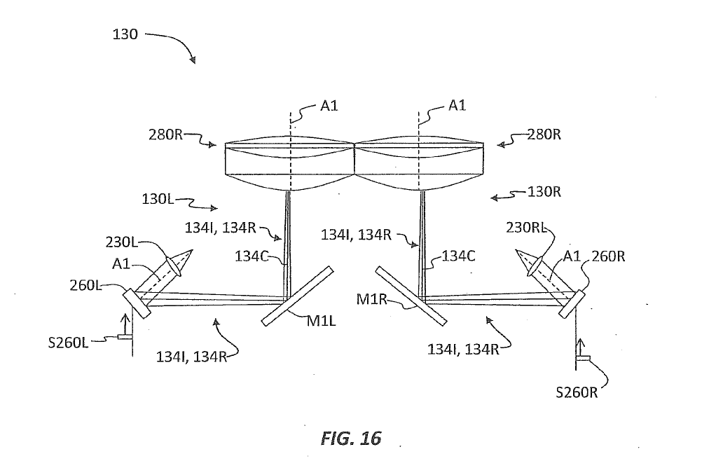

FIG. 16 is a schematic diagram of an exemplary configuration of the two (i.e., left and right) scanning optical systems 130L and 130R suitable for use in the dual scanning system 100 of FIG. 15.

FIG. 17 is schematic diagram similar to FIG. 15 that illustrates an exemplary embodiment of dual-head optical reader system 100 that uses only one set of one or more spectrometer units 140. This is accomplished by controller 150 and mirror device drivers 264 driving the respective scanning mirror devices 260 in an asynchronous manner so that only one set of reflected optical beams 134R (and thus one set of guided light signals 136) is processed by the one or more spectrometer units 140 at a time. In an example of this approach, signal S260L applied to scanning mirror 260L (see FIG. 16) is slightly offset (i.e., time-delayed) relative to signal S260R applied to scanning mirror device 260R. The consequence of this time delay is that, when scanning spot 135 associated left scanning optical system 130L is on a biosensor 102L, the scanning spot 135 (not shown in FIG. 17; see FIG. 8) associated with right scanning optical system 130R is in-between two biosensors 102R.

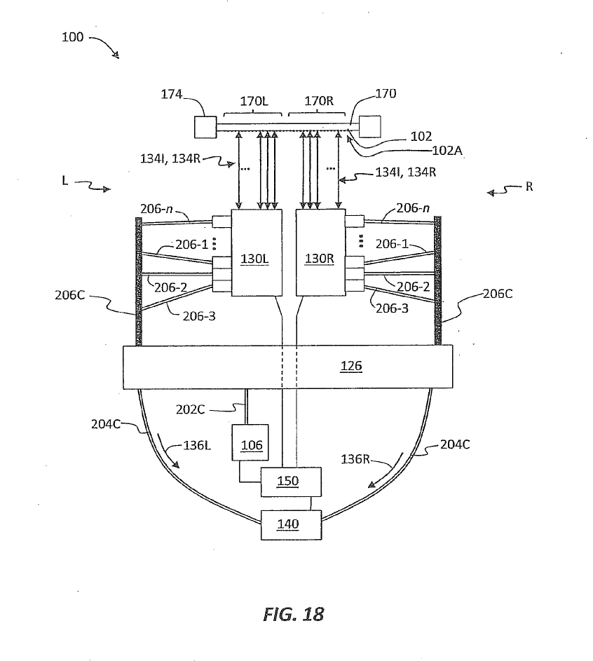

Consequently, while one scanning optical system 130 is generating a guided light signal 136, the other is generating no guided light signal. By interleaving the two guided light signals 136L and 136R, (e.g., via one or more coupling devices 126), and sending them to the one or more spectrometers 140 while tracking the delayed generation of scanning mirror signals S260L and 260R, the guided light signals from each scanning optical system and thus the corresponding spectrometer unit electrical signals 5140 are tracked. System 100 of FIG. 17 is shown configured with the various fiber sections 202, 204 and 206 in the form of optical fiber cables (e.g., ribbon cables) 202C, 204C and 206C that carry one or more of the respective optical fiber sections 202, 204 and 206. FIG. 18 is similar to FIG. 17 and illustrates a simplified example embodiment wherein system 100 includes a single coupling device 126, a single light source 160 and a single spectrometer unit 140.

While the dual-head optical reader optical system 100 can be more expensive to implement than the single-scanning optical reader, it is capable of making a relatively large number of scanned measurements of an array of biosensors 102 in a relatively short amount of time, e.g., a microplate 170 having an array of 16×24 biosensors 102 can be read in about 20 seconds. Dual-head optical reader systems can be particularly useful in high volume or high throughput scanning applications, such as in diagnostic methods or drug discovery methods.

### Data Processing Methods for RWG Biosensors

Variations in the position of light spot 135 on RWG biosensor 102 can act as noise that can degrade the precision and repeatability of the measurement of the resonant wavelength λ_{R}. Accordingly, an aspect of the disclosure includes improved data processing methods that reduce measurement noise and provide a more accurate measurement of the resonant wavelength λ_{R} for each scanned RWG biosensor 102.

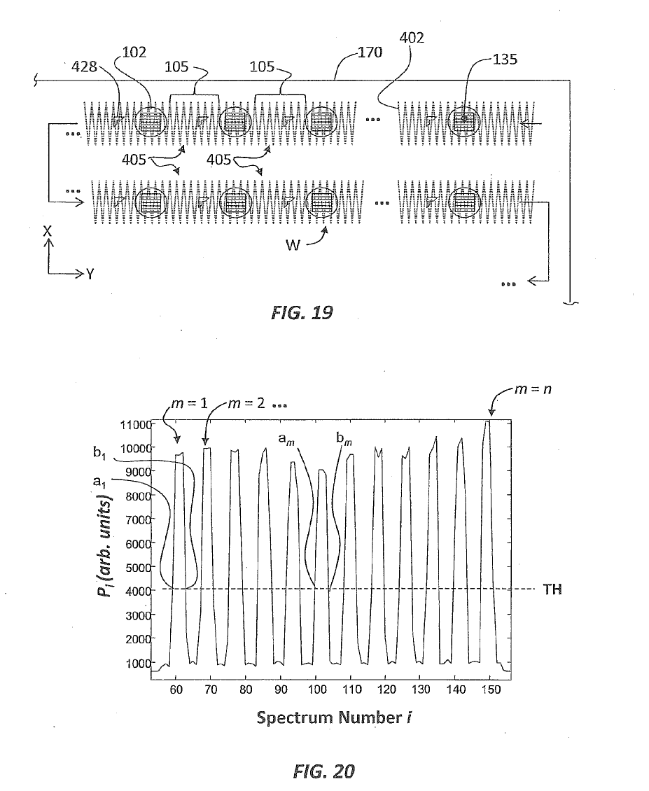

FIG. 19 is a close-up view of a section of microplate 170 (see also FIG. 2) that includes an example zig-zag scan path 402 for light spot 135. The example scan path 402 covers a select number of wells W, such as a 12×8 array portion of a 16×24 well array. In an example embodiment, scan path 402 is repeated over the remaining wells W on microplate 170 so that all of the RWG biosensors 102 are scanned. Other example zig-zag scan paths 402 can also be employed. Scan path 402 includes sections 405 that cover gaps 105 between RWG biosensors 102 where reflected light beam 134R does not include spectral information from a biosensor. Thus, the reflected light beams 134R collected during scanning result in saved data that includes series of spectra corresponding to individual RWG biosensors 102 separated by series of “spectra” from gaps 405 that contain no resonant-wavelength information.

Prior to calculating the resonant wavelength λ_{R} corresponding to individual RWG biosensors 102, in an example embodiment the spectral data is processed to determine where, in the series (i.e., set) of spectra saved during scanning, a given RWG biosensor starts and ends. This involves determining the position of the RWG bio sensor edges 102E.

RWG biosensor edge detection is based on the fact that individual wells W are separated by areas that have no RWG biosensors 102. The consequence is that, when incident beam 134I (and this light spot 135) is in-between wells W (i.e., within gaps 105) there is no resonance and the power reflected by microplate 170 is close to zero.

To detect when light spot 135 started hitting the edge 102E of a given RWG biosensor 102 during scanning, the detected power Pᵢ for each acquired spectrum is calculated. The detected power is defined as:

Pᵢ=∫Sᵢ(λ)dλ

where Pᵢ is the integrated power of the i^{th} spectrum, and Sᵢ (λ) is the i^{th} spectrum acquired during the scan of microplate 170.

FIG. 20 is a plot of the measured detector power Pᵢ vs. the spectrum number i. Pᵢ is also referred to as the integrated spectrum power, or just the “spectrum power.” The plot shows a series of peaks in the measured detector power Pᵢ that corresponds to the positions of RWG biosensors 102, while the valleys correspond to gaps 105 in between the biosensors. From the plot of FIG. 20, the positions of RWG biosensor edges 102E along the general direction of scan path 402 (i.e., the y-direction in FIG. 19) can be determined. In one example, this is accomplished by defining a vector a′ that contains a series of numbers, i.e., a′=(a′₁, a′₂, . . . ), corresponding to the spectrum number i where the spectrum power Pᵢ becomes larger than a select threshold value TH for the m^{th} spectrum. Likewise, a corresponding vector b′ is defined that contains a series of numbers, i.e., b′=(b′₁, b′₂, . . . ) corresponding to the spectrum number i where the spectrum power Pᵢ becomes smaller than the selected threshold value TH for the m^{th} spectrum.

In calculating the resonant wavelength λ_{R} for a given RWG biosensor 102, it is particularly useful that all of the spectra collected for the given biosensor are processed. One example method of ensuring this is to consider a slightly oversized area for the given RWG biosensor 102 by adding a number k of spectra on each side of RWG biosensor 102 to ensure that all spectra acquired for the associated well W are considered. The value of k is chosen to be small enough so that no spectra are taken from an adjacent RWG biosensor 102, but large enough so that all spectra for the particular RWG biosensor are collected. Thus, the two vectors a′ and b′ are modified to become:

a=a′−k=(a′₁l−k,a′₂−k, . . . a′ₘ−k, . . . a′ₙ−k)=(a₁,a₂, . . . aₘ, . . . aₙ))

b=b′+k=(b′₁+k,b′₂+k, . . . b′ₙ+k)=(b₁,b₂, . . . bₘ . . . bₙ)

FIG. 20 shows the values of a and b (namely, a₁ and b₁) for the m=1 portion of the plot of spectrum power Pᵢ vs. i.

The average spectrum <Sₘ(λ)> corresponding to the m^{th} RWG biosensor 102 of n measured biosensors is calculated as the sum of all i spectra from aₘ to bₘ, namely:

$$〈 {S}_{m} ( λ ) 〉 = {\sum }_{i = {a}_{m}}^{{b}_{m}} {{S}_{i} ( λ )}_{m}$$

where Sᵢ(λ)ₘ is the i^{th} spectrum acquired during the scanning of the m^{th} RWG biosensor 102.

When using a scanning mirror device 260 to scan light spot 135 over a scan path 402 over microplate 170 (see, e.g., FIG. 5) any noise in the mirror angle translates into positional errors of light spot 135 on the RWG biosensor 102 being scanned. Making matters worse is the fact that mirror angle variations result in amplified positional errors of light spot 135 due to the relatively large distance between scanning mirror device 260 and microplate 170. Consequently, one cannot accurately control the exact position of light spot 135 on RWG biosensor 102. Even when scanning mirror device 260 is driven using a constant signal from mirror device driver 264 (see FIG. 5), light spot 135 naturally moves (“vibrates”) around an average position.

As an example, when a MEMS-based mirror is selected for use in the scanning mirror device 260, any environmental excitation, such as vibrations or acoustic waves, can cause the MEMS mirror to vibrate at its natural resonant frequency. An example MEMS-mirror resonant frequency is about 120 Hz and the corresponding vibrational amplitude of light spot 135 at microplate 170 is about 30 micrometers. This vibration amplitude is much higher when optical reader system 120 is subject to external sources of vibration, such table-top vibrations caused by footsteps and other ambient sources of vibrations. The vibration of light spot 135 significantly increases the measurement noise in optical reader system 100, thereby reducing its performance.

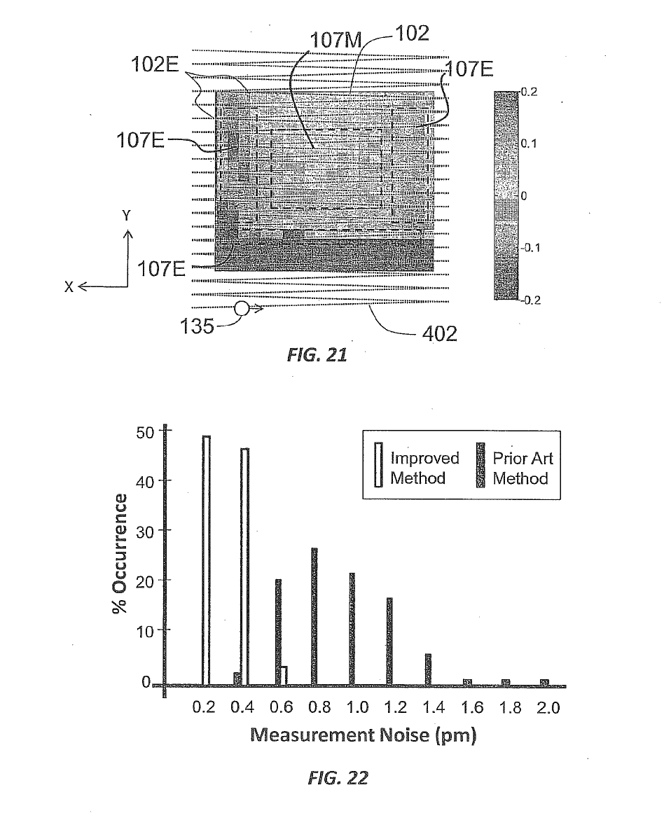

FIG. 21 is a two-dimensional gray-scale map of the resonant wavelength λ_{R} measured over an example RWG biosensor 102 as a function of position within the biosensor. An example portion of a zig-zag scan path 402 is shown for the sake of reference. As can be seen from the shading, the values for the resonant wavelength λ_{R} obtained in a middle portion 107M of the RWG biosensor 102 are reasonably uniform. Thus, in the middle portion 107M of RWG biosensor 102, some noise (i.e., variation in the position of light spot 135) is not expected to create a significant variation in the resonance wavelength λ_{R}.

However, the resonant wavelength λ_{R} tends to vary more as a function of position closer to edges 102E in edge portions 107E. Thus, when light spot 135 is within an edge portion 107E, the resonant wavelength λ_{R} can change significantly with a change in position of light spot 135. Consequently, any noise in scanning mirror device 260 has a larger impact on the measurement of the resonant wavelength λ_{R} when light spot 135 is close to a RWG biosensor edge 102E than when it is in the middle of the biosensor.

To minimize the effects that this light spot positional sensitivity has on the calculation of the resonant wavelength λ_{R}, the resonant wavelength values obtained at or close to RWG biosensor edges 102E in edge portions 107E are weighted less than those values obtained in the middle portion 107M of the RWG biosensor when calculating the resonant wavelength λ_{R} based on the average spectra <Sᵢ(λ)> in accord with the above equation. Equivalently, the spectra Sᵢ(λ)ₘ obtained in edge portions 107E are weighted less than spectra obtained in middle portions 107M. In respective examples, a spectrum in middle portion 107M can be weighted relative to a spectrum in at least one edge portion 107E by, for example, greater than 5%, greater than 10%, greater than 25%, greater than 50% and greater than 75%.

An example method of calculating the resonant wavelength λ_{R} is as follows. First, the average (center) position of RWG biosensor 102 is determined. In an example embodiment, the average position is taken in the general direction of scan path 402, which with reference to FIG. 19 is in the y-direction. In this direction, the typical zig-zag scan path 402 will cross each of the near and far edges 102E of RWG biosensor 102 only once while in the orthogonal (i.e., x-direction) it will cross each of the corresponding edges multiple times.

Thus, the average position <yₘ> of the m^{th} grating is calculated, for example, as the centroid of the spectra power, namely:

$$〈 {y}_{m} 〉 = {\sum }_{i = {a}_{m}}^{{b}_{m}} i {{P}_{i} ( λ )}_{m} / {\sum }_{i = {a}_{m}}^{{b}_{m}} {{P}_{i} ( λ )}_{m}$$

Next, a weighting function is used to calculate the average spectra, so that the equation for the average spectra <Sₘ(λ)> becomes:

$$〈 {S}_{m} ( λ ) 〉 = {\sum }_{i = {a}_{m}}^{{b}_{m}} A ( i ) {{S}_{i} ( λ )}_{m}$$

where A(i) is the weighting function, preferably centered on <yₘ>, that gives lower weight to spectra obtained near the edges 102E of RWG biosensor 102 (i.e., “edge spectra”). An example weighting function A(i) has the following form:

A(i)=exp{−pₙ(i−<yᵢₙ>}

where pₙ is a polynomial of degree n containing only even coefficients. Thus, an example weighting function is a Gaussian function. In certain other example embodiments, the weighting function is symmetrical, while in other example embodiments it is asymmetric. In an example embodiment, the weighting function A(i) is determined by examining the noise in resonant wavelength measurements and applying weighting values (“weights”) to the discrete measurement locations based on the value of the noise associated therewith. This allows for the weighting function to be tailored to compensate for certain noise signatures associated with RWG biosensor measurements.

An example Gaussian weighting is centered on the RWG biosensor and sets the full-width half-maximum (FWHM) of the Gaussian to cover a middle portion 107M that extends halfway to edges 102E. In another example, the +/−σ points of the centered Gaussian are located at halfway to edges 102E or alternatively at about ⅔ of the way to edges 102E. In another example, the +/−2σ points of the centered Gaussian are located halfway to edges 102E or alternatively at about ⅔ of the way to edges 102E. In another example, the 1/e² points of the centered Gaussian are located at edges 102E.

An example linear weighting weights the data such that the data half-way between the center and edge 102E is weighted by 50% less than the center, the data 80% of the distance between the center and the edge is weighted 80% less than at the center, etc.

FIG. 22 is a histogram of the % occurrence versus measurement noise (in picometers, pm) in the calculated resonant wavelength λ_{R} for measurements made on 96 RWG biosensors 102, with the resonant wavelength calculated using the prior art data processing method (black bars) and the improved data processing method (“improved method”) of the present disclosure (white bars). The histogram shows that the noise in the resonant wavelength measurements is fairly spread out, with a significant number of noise measurements of 1 pm and above, with the average being about 0.93 pm. The white histogram bars represent the noise measurements based on the data processing methods of calculating the resonant wavelength λ_{R} according to the present disclosure, with a Gaussian weighting function with the 1/e² points located at RWG biosensor edges 102E.

The noise measurements were reduced significantly to an average of about 0.31 pm, or by a factor of about 3× as compared to the prior art method. In an example embodiment, the method of the present disclosure reduces the noise in the resonant wavelength calculation by a factor of about 2× or greater (e.g., by between about 2× and 4×) as compared to using a non-weighted-average spectrum. In addition, the noise signature associated with the position of light spot 135 on the RWG biosensor (see FIG. 21) was eliminated.

In an example embodiment, the improved methods for calculating resonant wavelength λ_{R} are carried out by processor 152 according to instructions embodied in a computer-readable medium (e.g., in memory 154 or in the processor itself).

Various modifications to embodiment of the disclosure described herein can be made without departing from the spirit or scope of the disclosure as defined in the appended claims. Thus, the disclosure covers the modifications and variations provided they come within the scope of the appended claims and the equivalents thereto.

What is claimed is:

1. A method of measuring a resonant wavelength of a resonant waveguide (RWG) biosensor in an array of RWG biosensors supported by a microplate in a label-independent optical reader, comprising:
scanning a light spot over the RWG biosensor to obtain a plurality of spectra from a central portion and from at least one edge portion of the RWG biosensor;
calculating a weighted-average spectrum for the biosensor comprising averaging the plurality of spectra while applying greater weight to the central portion than to the at least one edge portion by greater than 5%; and
determining the resonant wavelength from the weighted-average spectrum.

2. The method of claim 1, further comprising determining an average position of the RWG biosensor.

3. The method of claim 2, wherein determining an average position of the RWG biosensor comprises:
calculating a power Pᵢ for each of the plurality of spectra; and
determining a centroid of the spectra powers Pᵢ as a function of i.

4. The method of claim 3, wherein calculating the weighted-average spectrum for the biosensor comprises applying a weighting function to the plurality of spectra.

5. The method of claim 4, further comprising centering the weighting function on the average position of the RWG biosensor.

6. The method of claim 4, wherein the weighting function includes an exponential function with even polynomial powers.

7. The method of claim 2, further comprising defining at least one edge location of the RWG biosensor including applying a select threshold value to the calculated power.

8. The method of claim 1, further comprising scanning the light spot over the RWG biosensor in a zig-zag scan path that crosses each of two opposing edges of the RWG biosensor multiple times.

9. The method of claim 1, wherein said calculating is caused to be carried out by a processor according to instructions embodied in a computer-readable medium.

10. A method of calculating a resonant wavelength of a resonant waveguide (RWG) biosensor having a central portion and at least one edge portion, based on a set of measured spectra obtained by scanning the RWG biosensor with a light beam and processing the reflected light, comprising:
determining an average position of the RWG biosensor;
calculating a weighted-average spectrum for the biosensor by averaging the set of spectra while applying a weighting function centered on the average position, the weighting function weights the central portion greater than the at least one edge portion by greater than 5%; and
calculating the resonant wavelength from the weighted-average spectrum.

11. The method of claim 10, wherein determining an average position of the RWG biosensor comprises:
calculating a power P for each of the plurality of spectra; and
determining a centroid of the spectra powers.

12. The method of claim 10, wherein calculating the resonant wavelength comprises finding a centroid of the weighted-average spectrum.

13. The method of claim 10, wherein the calculated resonant wavelength comprises less noise by a factor of at least two times compared to the resonant wavelength calculated using an unweighted-average spectrum.

14. The method of claim 10, wherein determining, calculating the spectrum, and calculating the wavelength, are accomplished by a processor according to instructions embodied in a computer-readable medium.

15. A method of reducing noise in a calculated resonant wavelength of a resonant waveguide (RWG) biosensor in an array of RWG biosensors supported by a microplate and each biosensor of the array being separated from any other biosensor by gaps, comprising:
scanning the plurality of RWG biosensors and the gaps therebetween with an optical beam and collecting reflected light from the biosensors and from the gaps;
establishing a set of spectra for each scanned RWG biosensor by calculating the spectral power of the reflected light and setting a power threshold that defines edge locations of the RWG biosensors;
calculating a weighted-average spectrum for each RWG biosensor comprising averaging the set of spectra for each RWG biosensor and applying a weighting function comprising weighting a central portion of the RWG biosensor more than edge portions of the RWG biosensor by greater than 5%; and
calculating the resonant wavelength from the weighted-average spectrum.

16. The method of claim 15, further comprising centering the weighting function at an average position of each biosensor.

17. The method of claim 15, further comprising determining the average positions of each of the RWG biosensors by calculating for each biosensor a spectrum power P for each of the plurality of spectra for the RWG biosensor, and determining a centroid of the spectrum powers for the RWG biosensor.

18. The method of claim 15, wherein calculating the resonant wavelength includes finding a centroid of the weighted-average spectrum.

19. The method of claim 15, further comprising accomplishing each of scanning, establishing, calculating the spectrum, and calculating the resonant wavelength with a processor according to instructions embodied in a computer-readable medium.

20. The method of claim 15, further comprising scanning the light spot over the RWG biosensor in a zig-zag scan path that crosses each of two opposing edges of the RWG biosensor multiple times.
